(19) United States
(12) Patent Application Publication
(10) Pub. No.: US 2023/0242945 A1
(43) Pub. Date: Aug. 3, 2023

(54) METHODS FOR PRODUCTION OF AMYELOIS TRANSITELLA PHEROMONE PRECURSOR

(71) Applicants: Jan Christer Lofstedt, Lund (SE); Per Fredrik Hofvander, Bjärred (SE); Honglei Wang, Lund (SE); Baojian Ding, Lund (SE); ISCA Technologies, Inc., Riverside, CA (US)

(72) Inventors: Jan Christer Löfstedt, Lund (SE); Per Fredrik Hofvander, Bjärred (SE); Honglei Wang, Lund (SE); Baojian Ding, Lund (SE); Agenor Mafra-Neto, Riverside, CA (US)

(21) Appl. No.: 18/161,794

(22) Filed: Jan. 30, 2023

Related U.S. Application Data
(60) Provisional application No. 63/305,121, filed on Jan. 31, 2022.

(51) Int. Cl. C12P 7/04; C12N 9/02

(52) U.S. Cl. CPC C12P 7/04; C12N 9/0008; C12N 9/0071; C12Y 114/19005

(57) ABSTRACT

The present invention relates to methods of producing Amyelois transitella pheromone precursors and genetically modified plants and microorganisms capable of producing Amyelois transitella pheromone precursors. The genetically modified plants and microorganisms include a heterologous gene encoding a first fatty-acyl desaturase and a second fatty-acyl desaturase in combination, and a fatty-acyl reductase.

### CROSS-REFERENCE TO RELATED APPLICATIONS

This application claims the benefit of U.S. Provisional Application No. 63/305,121, filed on Jan. 31, 2022, the teachings of which are both expressly incorporated by reference.

### STATEMENT RE: FEDERALLY SPONSORED RESEARCH/DEVELOPMENT

Not Applicable

### BACKGROUND

### Technical Field

The present disclosure relates generally to a process of preparation of insect pheromones in plants and microorganisms, and more particularly, insect pheromones of Amyelois transitella in plants and microorganisms.

### Description of the Prior Art

Amyelois transitella (navel orangeworm) belongs to the order of Lepidoptera, family Pyralidae and it is a major pest of walnut, fig, almond, pistachio, and a number of other crops.

Insecticidal control of Amyelois transitella is not efficient because the larval and pupal stages are protected inside the plant, and also because all developmental stages are present throughout the year, but integrated pest management (IPM), where the focus lies on monitoring, prevention and limited use of pesticides, optimised cultural practices using resistant cultivars, and biological control or the use of sex pheromones may provide a solution.

Pheromones are environmentally friendly alternatives to the use of traditional pesticides for the control of insect pests. For this purpose, synthetic pheromones are produced annually in large quantities. The use of pheromones for the control of pest insects has many advantages over the use of conventional chemical-based pesticides.

These pheromones are non-toxic. They have no adverse effects on non-target organisms, and do not kill parasitoids or other beneficial insects. The risks of resistance being developed in the pests are small. Even in terms of profit and reduction in damage, pheromones often compare favorably to the use of insecticides. In the case of treating cabbage against diamondback moth infestation, pheromone-based integrated pest management was found to be inexpensive ($62 relative to $123 per ha) resulting in a higher gross profit (ca $800 compared to $456 per ha) in comparison to the conventional practice with insecticides. The global market for pheromone-based control products is currently estimated to be approximately $200 million.

Amyelois transitella uses two types of sex pheromone components, type I pheromone components, one of which is Z11,Z13-hexadecadienal (Z11,Z13-16:Ald) and type II pheromone components including the unusual long-chain pentaenes, Z3,Z6,Z9,Z12,Z15-tricosapentaene and Z3,Z6,Z9,Z12,Z15-pentacosapentaene.

Studying how sex pheromones are biosynthesised by the insects is critical for development of biotechnological pheromone production to obtain the active compounds needed for pest management, and the successful use of insect enzymes for production of moth pheromones in yeasts and plant have been demonstrated. Moth sex pheromone biosynthesis involves genes in the large multigene families of fatty acyl desaturases (desaturases) and fatty acyl reductases (reductases), fatty alcohol oxidases and acetyltransferases. The biosynthesis of conjugated lepidopteran sex pheromones similar to Z11,Z13-16:Ald has been described in moths from several families. The main pheromone components of Lampronia capitella (Prodoxidae) (9Z,11Z)-tetradecadienol (Z9,Z11-14:OH), Epiphyas postvittana (Tortricidae) (9E, 11E)-tetradecadienyl acetate (E9,E11-14:OAc), and Spodoptera litura (Noctuidae) (9Z,11E)-tetradecadienyl acetate (Z9,E11-14:OAc) are made by one desaturase belonging to the clade of specific lepidopteran Δ11-desaturases, making both double bonds in sequence with a chain-shortening step in between. The major pheromone compounds of Cydia pomonella (Tortricidae) (8E,10E)-dodecadienol (E8,E10-12:OH) and Bombyx mori (Bombycidae) (10E,12Z)-hexadecadienol (E10,Z12-16:OH) are made by bifunctional desaturases that first introduce one double bond in the intermediate position and then turn this into the conjugated diene pheromone component. Two desaturases and several chain-shortening steps are involved in the biosynthesis of a pheromone component of Dendrolimus punctatus (Lasiocampidae), (5Z,7Z)-dodecadienol (Z5,Z7-12:OH), either using a desaturase homologous to common 49 acyl-CoA desaturases and a Lepidoptera specific Δ11-desaturase, or two Δ11-desaturases. The biosynthesis of the (7E,9Z)-dodecadienyl acetate (E7,Z9-12:OAc) pheromone component of Lobesia botrana (Tortricidae) involves a Δ11-desaturase and chain-shortening followed by the action of an elusive desaturase that makes a Δ7 double bond in the Z9-12:acyl. The biosynthetic pathways of these diunsaturated sex pheromone components may serve as hypotheses for Amyelois transitella biosynthesis of Z11,Z13-16:Ald.

As such, there is a need for improved methods with increased production of Amyelois transitella pheromones and their precursors in plant or microbe factories.

### BRIEF SUMMARY

The present invention demonstrates the feasibility of the production of large amounts of insect (moth) pheromone precursors of Amyelois transitella insects.

In accordance with one aspect, the invention relates to relates to a genetically modified plant having incorporated into its genome a heterologous gene(s) encoding a first fatty-acyl desaturase and a second fatty-acyl desaturase in combination, and a fatty-acyl reductase, wherein the plant produces an Amyelois transitella pheromone precursor.

In accordance with another aspect, the invention relates to a genetically modified microorganism having incorporated into its genome a heterologous gene(s) encoding a first fatty-acyl desaturase and a second fatty-acyl desaturase in combination, and a fatty-acyl reductase, wherein the microorganism produces at least one Amyelois transitella pheromone precursor.

In accordance with yet another aspect, the invention relates to a method of producing Amyelois transitella pheromone precursors. The method involves selecting a plant or a microorganism to be genetically modified, incorporating into its genome, a heterologous gene encoding a first fatty-acyl desaturase and a second fatty-acyl desaturase in combination, and a fatty-acyl reductase to obtain a genetically modified plant or a genetically modified microorganism, and producing Amyelois transitella pheromone precursors from the genetically modified plant or the genetically modified microorganism.

By way of this invention, it is for the first time that it has been made possible to produce Amyelois transitella pheromone precursor and therefrom Amyelois transitella pheromones.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates a pathway for biosynthesis of Amyelois transitella pheromone component fatty alcohol precursor;

FIG. 2 illustrates functional characterization of Amyelois transitella candidate desaturases and reductase in a Nicotiana benthamiana expression system; and

FIG. 3 illustrates a chromatogram of Soxhlet extraction from Nicotiana benthamiana expression system.

### DETAILED DESCRIPTION

The detailed description set forth below is intended as a description of the presently preferred embodiment of the invention and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

### Definitions

In the context of the present application and invention, the following definitions apply:

As used herein, the terms “microbial,” “microbial organism,” and “microorganism” include any organism that exists as a microscopic cell that is included within the domains of archaea, bacteria or eukarya, the latter including yeast and filamentous fungi, protozoa, algae, or higher Protista. Therefore, the term is intended to encompass prokaryotic or eukaryotic cells or organisms having a microscopic size and includes bacteria, archaea, and eubacteria of all species as well as eukaryotic microorganisms such as yeast and fungi. Also included are cell cultures of any species that can be cultured for the production of a chemical.

The term “genetic modification” implies the introduction of homologous and/or heterologous foreign nucleic acid molecules into the genome of a plant cell or into the genome of a microorganism, wherein said introduction of these molecules leads to an accumulation of insect pheromone precursors.

The term “recombinant microorganism” and “genetically modified microorganism” are used interchangeably herein and refer to microorganisms that have been genetically modified to express or to overexpress endogenous enzymes, to express heterologous enzymes, such as those included in a vector, in an integration construct, or which have an alteration in expression of an endogenous gene.

The term “expression” with respect to a gene sequence refers to transcription of the gene and, as appropriate, translation of the resulting mRNA transcript to a protein.

The term “polynucleotide” is used herein interchangeably with the term “nucleic acid” and refers to an organic polymer composed of two or more monomers including nucleotides, nucleosides or analogs thereof, including but not limited to single stranded or double stranded, sense or antisense deoxyribonucleic acid (DNA) of any length and, where appropriate, single stranded or double stranded, sense or antisense ribonucleic acid (RNA) of any length, including siRNA.

The term “enzyme” as used herein refers to any substance that catalyzes or promotes one or more chemical or biochemical reactions, which usually includes enzymes totally or partially composed of a polypeptide or polypeptides but can include enzymes composed of a different molecule including polynucleotides.

The term “exogenous” as used herein with reference to various molecules, e.g., polynucleotides, polypeptides, enzymes, etc., refers to molecules that are not normally or naturally found in and/or produced by a given yeast, bacterium, organism, microorganism, or cell in nature.

On the other hand, the term “endogenous” or “native” as used herein with reference to various molecules, e.g., polynucleotides, polypeptides, enzymes, etc., refers to molecules that are normally or naturally found in and/or produced by a given yeast, bacterium, organism, microorganism, or cell in nature.

The term “heterologous” as used herein describes a relationship between two or more elements which indicates that the elements are not normally found in proximity to one another in nature. Thus, for example, a polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g., a genetically engineered coding sequence or an allele from a different ecotype or variety). An example of a heterologous polypeptide is a polypeptide expressed from a recombinant polynucleotide in a transgenic organism. Heterologous polynucleotides and polypeptides are forms of recombinant molecules.

The term “fatty acid” as used herein refers to a compound of structure R—COOH, wherein R is a C₆ to C₂₄ saturated, unsaturated, linear, branched or cyclic hydrocarbon and the carboxyl group is at position 1.

The term “fatty alcohol” as used herein refers to an aliphatic alcohol having the formula R—OH, wherein R is a C₆ to C₂₄ saturated, unsaturated, linear, branched or cyclic hydrocarbon.

The term “fatty acyl-CoA” refers to a compound having the structure R—(CO)—S—R₁, wherein R₁ is Coenzyme A.

### Plant Platforms for Pheromone Production

In this disclosure, two plant platforms were utilized: Nicotiana benthamiana and Camelina sativa for pheromone production.

N. benthamiana is a close relative of N. tabacum, the most commonly grown commercial plant in the Nicotiana genus for its leaves to produce tobacco. Mature plants usually show a large variation in height, ranging from as tall as 1.5 meters to shorter than 200 mm. The Nicotiana species is favorable to work with in metabolic engineering aiming at production of pheromone compounds as they have relatively short production times, large area of leaves to output volatiles and are relatively easier to grow in controlled growth conditions. In addition, there is less concern about contaminating food supplies as they are not food crops.

Camelina was chosen as the oilseed production platform because it has limited use as a food crop and is considered an ideal system for rapid introduction and evaluation of fatty acid and other oil-related traits. Further, transgenes can easily be introduced into Camelina using a simple Agrobacterium-based method, and it has a relatively short life cycle that allows up to three generations in a year for evaluation of engineered traits. Camelina is also closely related to Arabidopsis thaliana, with a wealth of transgenic and genomic data for optimizing endogenous biosynthetic pathways for production of desired oil traits in seeds that typically are 30% to 40% oil by weight.

### Biosynthesis of Pheromones Using a Genetically Modified Plant

As discussed above, in a first aspect, the present invention relates to a genetically modified plant having incorporated into its genome a heterologous gene(s) encoding a first fatty-acyl desaturase and a second fatty-acyl desaturase in combination, and a fatty-acyl reductase, wherein the plant produces a Amyelois transitella pheromone precursor. In an embodiment, the first fatty-acyl desaturase is a 411 desaturase and the second fatty-acyl desaturase is a 413 desaturase.

An exogenous fatty acyl desaturase described herein can be selected to catalyze the desaturation at a desired position on the hydrocarbon chain. Accordingly, in some embodiments, a Δ11 desaturase is capable of generating a double bond at C₁₁ position and Δ13 desaturase at C₁₃ position in the fatty acid or its derivatives, such as, for example, fatty acid CoA esters.

The major female sex pheromone component of A. transitella has been identified as (11Z,13Z)-hexadecadienal (Z11,Z13-16:Ald).

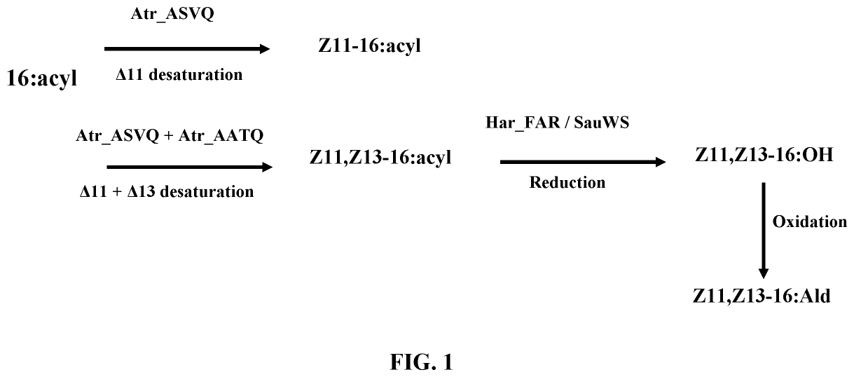

FIG. 1 illustrates a pathway for biosynthesis of Amyelois transitella pheromone component fatty alcohol precursor.

The present invention explores the production of pheromone precursors in their fatty alcoholic form which on oxidation can be readily converted to the aldehydes which are the pheromones.

In one embodiment, the first fatty-acyl desaturase and the second fatty-acyl desaturase in combination catalyze the conversion of a C₁₆ fatty-acyl-CoA to a di-unsaturated C₁₆ fatty-acyl-CoA.

In one embodiment, the first fatty-acyl desaturase and the second fatty-acyl desaturase generate a double bond at the C₁₁ position and C₁₃ position, respectively. In an exemplary embodiment, the first fatty-acyl desaturase is Atr_ASVQ, a desaturase obtained from A. transitella. In another exemplary embodiment, the second fatty-acyl desaturase is Atr_AATQ, also obtained from A. transitella.

In one embodiment, the fatty-acyl reductase catalyzes the conversion of the di-unsaturated C₁₆ fatty-acyl-CoA to a di-unsaturated C₁₆ fatty alcohol. In an exemplary embodiment, the fatty-acyl reductase is one selected from the group consisting of Har_FAR and SauWS.

The di-unsaturated C₁₆ fatty alcohol can be further oxidized to a di-unsaturated C₁₆ fatty aldehyde.

### Biosynthesis of Pheromones Using a Genetically Modified Microorganism

In a second aspect, the present invention relates to a genetically modified microorganism having incorporated into its genome a heterologous gene(s) encoding a first fatty-acyl desaturase and a second fatty-acyl desaturase in combination, and a fatty-acyl reductase, wherein the microorganism produces at least one Amyelois transitella pheromone precursor. In an embodiment, the first fatty-acyl desaturase is a Δ11 desaturase and the second fatty-acyl desaturase is a Δ13 desaturase.

In one embodiment, the first fatty-acyl desaturase and the second fatty-acyl desaturase in combination catalyze the conversion of a C₁₆ fatty-acyl-CoA to a di-unsaturated C₁₆ fatty-acyl-CoA.

In one embodiment, the first fatty-acyl desaturase and the second fatty-acyl desaturase in combination generate a double bond at the C₁₁ position and C₁₃ position, respectively. In an exemplary embodiment, the first fatty-acyl desaturase is Atr_ASVQ, a desaturase obtained from A. transitella. In another exemplary embodiment, the second fatty-acyl desaturase is Atr_AATQ, again obtained from A. transitella.

In one embodiment, the fatty-acyl reductase catalyzes the conversion of the di-unsaturated C₁₆ fatty-acyl-CoA to a di-unsaturated C₁₆ fatty alcohol. In an exemplary embodiment, the fatty-acyl reductase is one selected from the group consisting of Har_FAR and SauWS.

The di-unsaturated C₁₆ fatty alcohol can be further oxidized to at least one saturated, mono-, or di-unsaturated C₁₆ fatty aldehyde.

In an embodiment, the microorganism is yeast. In an exemplary embodiment, the yeast is Saccharomyces cerevisiae.

Method of Biosynthesis of Pheromones Using a Genetically Modified Plant or Microorganism

In a third aspect, the present invention relates to a method of producing Amyelois transitella pheromone precursors. The method involves selecting a plant or a microorganism to be genetically modified, incorporating into its genome, a heterologous gene encoding a first fatty-acyl desaturase and a second fatty-acyl desaturase in combination, and a fatty-acyl reductase to obtain a genetically modified plant or a genetically modified microorganism, and producing Amyelois transitella pheromone precursors from the genetically modified plant or the genetically modified microorganism.

In an embodiment, the method involves catalyzing, by the first and the second fatty-acyl desaturases in combination, conversion of a C₁₆ fatty-acyl-CoA to a di-unsaturated C₁₆ fatty-acyl-CoA. In another embodiment, the method involves catalyzing, by the fatty-acyl reductase, conversion of the di-unsaturated C₁₆ fatty-acyl-CoA into an Amyelois transitella pheromone precursor. In yet another embodiment, the method further involves oxidizing the Amyelois transitella pheromone precursor to an Amyelois transitella pheromone.

### Pheromones

In different conditions, pheromones made using the invention's techniques and compositions including the pheromones can be employed to regulate Amyelois transitella insect behaviour and/or development. For instance, the pheromones can be employed to draw male Amyelois transitella insects to or away from a certain target region. Pheromones can be employed to draw Amyelois transitella insects away from agricultural regions that are particularly vulnerable. Insect monitoring, mass capturing, lure/attract-and-kill, or mating disruption strategies can all be utilised with the pheromones to draw in insects.

### Lures

In accordance with the embodiments of the present invention, lures may be coated with, sprayed with, or otherwise impregnated with one of the pheromone compositions described in the current disclosure.

### Traps

The pheromone compositions described in the disclosure may be employed in traps that are often used to draw Amyelois transitella insects. Such traps are widely utilised in many states and nations in pest eradication projects and are well recognised to those competent in the field. For retaining the pheromone mixture, the trap in one embodiment has one or more septa, containers, or storage receptacles. Thus, the current disclosure offers a trap that is loaded with at least one pheromone compound in certain embodiments. The pheromone compositions of the current disclosure can therefore be utilised in traps, for example, to entice Amyelois transitella insects as part of an approach for insect monitoring, mass trapping, mating disruption, or lure/attract and kill, for example, by incorporating a toxic substance into the trap to kill Amyelois transitella insects caught.

### Mating Disruption

Pheromones made using the disclosed techniques can also be utilised to interfere with mating. Introducing artificial stimuli (such as the pheromone composition given here) that confuses the insects and disturbs mating location and/or courting prevents mating and stops the reproductive cycle. This approach is known as mating disruption and is used to control insect infestations.

### Attract and Kill

The attract and kill approach, which can have the same results as mass-trapping, uses an attractant, such as a sex pheromone, to entice insects of the target species to an insecticidal chemical, surface, gadget, etc. for mass death and ultimate population reduction. When a synthetic female sex pheromone is used to attract male pests, such as moths, in an attract-and-kill technique, for example, a significant number of male moths must be killed over a prolonged period of time in order to restrict mating and reproduction and ultimately control the pest population.

In the following section, the aspect is described by way of examples to illustrate the processes of the invention. However, these do not limit the scope of the present invention. Several variants of these examples would be evident to persons ordinarily skilled in the art.

### EXAMPLES

### Example 1

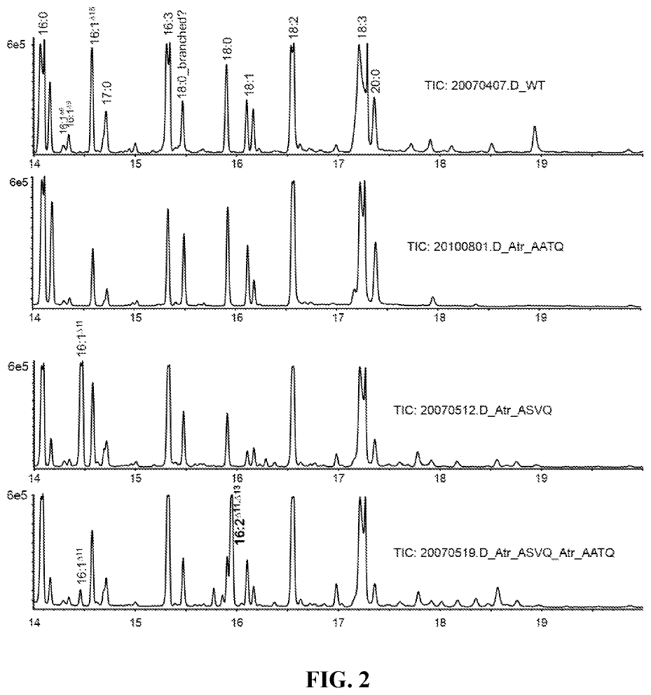

Atr_ASVQ, Atr_AATQ and Har_FAR were transiently expressed in Nicotiana benthamiana. FIG. 2 illustrates that a combination of Atr_ASVQ and Atr_AATQ was efficient in producing Z11,Z13-hexadecenoic acid (Z11,Z13-16: acid). The obtained Z11,Z13-hexadecenoic acid (Z11,Z13-16:acid) was further reduced by Har_FAR to Z11,Z13-hexadecadienol (Z11,Z13-16:OH).

### Example 2

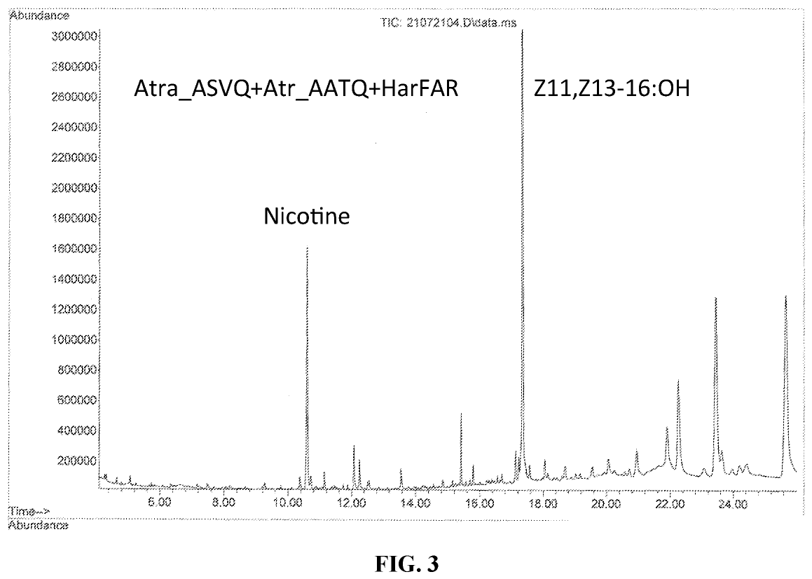

By agrobacterium infiltration of Atr_ASVQ, and Atr_AATQ and a fatty acyl reductase Har_FAR, Z11,Z13-16:OH was produced in Nicotiana benthamiana leaves. The Z11,Z13-16:0H was extracted using Soxhlet extraction and oxidized to final pheromone Z11,Z13-16:Aldehyde. FIG. 3 illustrates a chromatogram of the Soxhlet extraction. It indicates the production of Z11,Z13-16:OH.

### Example 3

Seeds of Camelina sativa were genetically modified for the production of Amyelois transitella pheromone precursor, Z11,Z13-16:acid. The gene expression was incorporated into triacylglycerol in the seeds. T2 generation is harvested. It is expected that T5 lines will be able to produce Z11,Z13-16:acid in a large scale. If Atr_ASVQ, and Atr_AATQ are combined with Har_FAR and SauWS, the Z11,Z13-16:OH can be incorporated into wax esters.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A genetically modified plant having incorporated into genome thereof, a heterologous gene encoding:
a) a first fatty-acyl desaturase and a second fatty-acyl desaturase in combination; and
b) a fatty-acyl reductase;
wherein the genetically modified plant produces at least one Amyelois transitella pheromone precursor.

2. The genetically modified plant of claim 1, wherein the first fatty-acyl desaturase is a Δ11 desaturase and the second fatty-acyl desaturase is a Δ13 desaturase.

3. The genetically modified plant of claim 1, wherein the first fatty-acyl desaturase and the second fatty-acyl desaturase in combination catalyze the conversion of a C₁₆ fatty-acyl-CoA to a di-unsaturated C₁₆ fatty-acyl-CoA.

4. The genetically modified plant of claim 3, wherein the fatty-acyl reductase catalyzes the conversion of the di-unsaturated C₁₆ fatty-acyl-CoA to a di-unsaturated C₁₆ fatty alcohol.

5. The genetically modified plant of claim 4, wherein the di-unsaturated C₁₆ fatty alcohol is oxidized to a di-unsaturated C₁₆ fatty aldehyde.

6. The genetically modified plant of claim 2, wherein the first fatty-acyl desaturase is Atr_ASVQ, and the second fatty-acyl desaturase is Atr_AATQ.

7. The genetically modified plant of claim 6, wherein the first fatty-acyl desaturase and the second fatty-acyl desaturase in combination catalyze the conversion of a C₁₆ fatty-acyl-CoA into a Z11,Z13:C₁₆ fatty-acyl-CoA.

8. The genetically modified plant of claim 1, wherein the fatty-acyl reductase is Har_FAR or SauWS.

9. The genetically modified plant of claim 4, wherein the di-unsaturated C₁₆ fatty alcohol is (Z,Z)-11,13-hexadecadienol.

10. The genetically modified plant of claim 1, wherein the plant is Nicotiana benthamiana or Camelina sativa.

11. A genetically modified microorganism having incorporated into genome thereof, a heterologous gene encoding:
a) a first fatty-acyl desaturase and a second fatty-acyl desaturase in combination; and
b) a fatty-acyl reductase;
wherein the genetically modified microorganism produces at least one Amyelois transitella pheromone precursor.

12. The genetically modified microorganism of claim 11, wherein first fatty-acyl desaturase is a Δ11 desaturase and the second fatty-acyl desaturase is a Δ13 desaturase.

13. The genetically modified microorganism of claim 11, wherein the first fatty-acyl desaturase and the second fatty-acyl desaturase in combination catalyze the conversion of a C₁₆ fatty-acyl-CoA to a di-unsaturated C₁₆ fatty-acyl-CoA.

14. The genetically modified microorganism of claim 13, wherein the fatty-acyl reductase catalyzes the conversion of the di-unsaturated C₁₆ fatty-acyl-CoA into a di-unsaturated C₁₆ fatty alcohol.

15. The genetically modified microorganism of claim 14, wherein the at least one saturated, mono- or di-unsaturated C₁₆ fatty alcohol is oxidized to at least one saturated, mono-, or di-unsaturated C₁₆ fatty aldehyde.

16. The genetically modified microorganism of claim 12, wherein the first fatty-acyl desaturase is Atr_ASVQ, and the second fatty-acyl desaturase is Atr_AATQ.

17. The genetically modified microorganism of claim 16, wherein the first fatty-acyl desaturase and the second fatty-acyl desaturase in combination catalyze the conversion of a C₁₆ fatty-acyl-CoA into a Z11,Z13:C₁₆ fatty-acyl-CoA.

18. The genetically modified microorganism of claim 11, wherein the fatty-acyl reductase is Har_FAR or SauWS.

19. The genetically modified microorganism of claim 14, the di-unsaturated C₁₆ fatty alcohol is (Z,Z)-11,13-hexadecadienol.

20. The genetically modified microorganism of claim 1, wherein the microorganism is a yeast.

21. The genetically modified microorganism of claim 20, wherein the yeast is Saccharomyces cerevisiae.

22. A method of producing Amyelois transitella pheromone precursors, said method comprising:
a) selecting a plant or a microorganism to be genetically modified;
b) incorporating into the genome thereof, a heterologous gene encoding a first fatty-acyl desaturase and a second fatty-acyl desaturase in combination, and a fatty-acyl reductase to obtain a genetically modified plant or a genetically modified microorganism; and
c) producing Amyelois transitella pheromone precursors from the genetically modified plant or the genetically modified microorganism.

23. The method of claim 22 comprising catalyzing, by the first and the second fatty-acyl desaturases in combination, conversion of a C₁₆ fatty-acyl-CoA to a di-unsaturated C₁₆ fatty-acyl-CoA.

24. The method of claim 23 comprising catalyzing, by the fatty-acyl reductase, conversion of the di-unsaturated C₁₆ fatty-acyl-CoA into an Amyelois transitella pheromone precursor.

25. The method of claim 24 comprising oxidizing the Amyelois transitella pheromone precursor to an Amyelois transitella pheromone.
